(19) United States
(12) Patent Application Publication
(10) Pub. No.: US 2008/0171958 A1
(43) Pub. Date: Jul. 17, 2008

(54) Wound Dressing

(76) Inventor: Boerge Gundersen, Tikoeb (DK)

Correspondence Address: JACOBSON HOLMAN PLLC, 400 SEVENTH STREET N.W., SUITE 600, WASHINGTON, DC, 20004, US

(21) Appl. No.: 11/885,049

(22) PCT Filed: Feb. 22, 2006
(86) PCT No.: PCT/DK2006/000104

§ 371 Date: Aug. 24, 2007

(30) Foreign Application Priority Data
Feb. 24, 2005 (DK) PA 2005 00280

(51) Int. Cl. A61F 13/02; A61F 13/15

(52) U.S. Cl. 602/56; 602/58

(57) ABSTRACT

A wound dressing comprising a backing layer and a skin facing layer and an absorbent pad, wherein the absorbent pad is sandwiched between the backing layer and the skin facing layer, and the two layers constitutes an envelope, the absorbent pad has an expansion of surface area, when fully expanded, of at least 10%, and wherein the surface area of said envelope is at least 10% larger than the surface area of the non-expanded absorbent pad. The envelope provides room for expansion of the absorbent pad without buckling or folding of the absorbent pad.

### BACKGROUND OF THE INVENTION

1. Field of the Invention

The invention relates to wound dressings, especially wound dressings for exuding wounds.

2. Description of the Related Art

In the treatment of chronic wounds such as leg ulcers and pressure sores it is often a problem that the skin surrounding the wound is extremely fragile and susceptible to develop further sores when exposed to irregularities in the dressing such as wrinkles and folds, or even a sharp edge of dressing or dressing element.

Furthermore, maceration is often a problem in the treatment of highly exuding wounds. The absorbent layer of the dressing absorbs exudates, but may distribute the exudates lateral from the wound, thus exposing the healthy, but fragile skin surrounding the wound to moisture, and thereby inducing maceration. Dressings worn vertically, such as dressings for leg ulcers, may often suffer from maceration of the skin beneath the wound and leakage, due to the gravity forces the exudates to seek downwards.

Dressings for chronic wounds typically comprise an absorbent element in the form of a layer or pad of an absorbent material, such as foam, alginate or other suitable absorbent materials. The absorbent element may be provided with an adhesive layer and a backing layer, often in the form of an island dressing. When such materials absorb liquid they may expand in volume, sometimes more than 40-100% v/v. As the absorbent element is “trapped” by being attached to a backing and/or adhesive layer or a secondary dressing that does not expand in the same rate as the absorbent element, the absorbent element may buckle or fold, thus giving rise to pressure sores as well as leakage and maceration.

When using foam as an absorbent element, pressure marks may appear if the foam expansion results in wrinkles and double layer foam at the edge. This happens when the foam is not allowed to expand freely, and there is no space left for expansion. If the foam expansion does not end up in a double or even triple layer at the edge, the foam will instead expand towards the skin and wound and this often results in pressure marks too.

The expanding foam causes stress in the entire product design, which results in the loosening of the adhesion from the skin, which may cause leakage or the dressing to detach from the skin.

In German patent application No. 100 54 928 is disclosed a method of producing an absorbent wound dressing wherein super absorbent particles are coated on a film in a pattern, leaving room for expansion into non-coated areas.

US patent application No. 2004/0127831 discloses a wound dressing comprising an absorbent element, e.g. in the form of a foam, an adhesive layer and a backing layer. The backing layer is provided with a concentric ridge in the form of folded film, which can be unfolded during expansion of absorbent layer, thus leaving room for the absorbent element to expand vertically. Lateral expansion of the absorbent layer is difficult because the adhesive layer immobilizes the absorbent layer and undesired stress will be built up in the dressing.

Thus there is still a need for a wound dressing being capable of handling an expanding absorbent element without giving rise to pressure marks or sores.

### SUMMARY OF THE INVENTION

One object of the present invention is to provide a wound dressing where the absorbent element is free to expand.

Another object of the invention is to provide a wound dressing being less susceptible to induce pressure sores.

Yet another object of the invention is to provide a dressing that does not buckle or unintended detach from the skin when wetted, but stays flat and smooth.

Yet another object of the invention is to provide a dressing without inherent tensions and stress.

Still another object of the present invention is to provide a dressing being capable of controlling wound exudates while in a vertical position.

Yet another object of the invention is to provide a dressing for preventing maceration of the skin surrounding the wound.

### BRIEF DESCRIPTION OF THE DRAWINGS

The invention is disclosed more in detail with reference to the drawings in which

FIG. 1 discloses an adhesive embodiment of the present invention,

FIG. 2 shows another adhesive embodiment of the invention,

FIG. 3 shows a non-adhesive embodiment of the present invention and

FIG. 4 shows a preferred non-adhesive embodiment of the invention.

### DETAILED DESCRIPTION OF THE PRESENT INVENTION

The invention relates to a wound dressing comprising a backing layer and a skin facing layer and an absorbent pad, wherein the absorbent pad is sandwiched between the backing layer and the skin facing layer, and the two layers constitutes an envelope, the absorbent pad has an expansion of surface area, when fully expanded, of at least 10%, and wherein the surface area of said envelope is at least 10% larger than the surface area of the non-expanded absorbent pad.

The envelope constitutes of a backing layer and a skin-facing layer. The two layers are preferably sealed along the edge portion or near the edge portion to obtain an envelope or bag. The two layers may have approximately the same size and shape or one of the layers may be larger than the other. The backing layer may e.g. be larger and extend further than the edges of the skin-facing layer, thus providing a flange around the envelope. When addressing the lateral dimensions of the envelope there is referred to the distance between the sealed edge portions, the closed volume of the envelope. The part of one or more of the two layers that may be extending further than the seal is not to be considered in the measurement.

By incorporating the absorbent pad in an envelope being large enough to handle the expansion of the absorbent pad, when the pad is wetted, and facilitate that the absorbent pad may move freely or float inside the envelope, the problem of pressure sores caused by folding and wrinkling of a swollen absorbent pad has surprisingly been eliminated. Pressure marks arising from double layer of foam are prevented by allowing the pad to expand in the lateral direction, thus avoiding expansion only in the vertical direction towards the wound/skin.

The ability of the absorbent pad to float freely in the envelope renders it possible to obtain a flat dressing without undesired tensions, which may induce discomfort and even pressure sores. When wetted, the pad may expand and slide in the envelope into the excess room of the envelope.

The surface area of the envelope is measured as the inside area between the edge seals, e.g. determined by the lateral dimensions.

It is preferred that the absorbent pad is capable of expanding freely or almost freely in the envelope. If the absorbent pad is allowed to expand freely, it does not cause stress in the remaining product design, including the adhesive surface towards the skin. Thus prevents adhesive to loosen from the skin and prevent leakage.

The surface area of the envelope may be at least 20% larger than the surface area of the absorbent pad, more preferred at least 30%, even more preferred 40% larger than the surface area of the absorbent pad.

In one embodiment of the invention the surface area of the envelope is at least 50%, more preferred 60% even more preferred 70% and even most preferred 80% larger than the surface area of the absorbent pad. The surface area of the envelope may be at least 100% larger than the surface area of the absorbent pad.

If the envelope allows the absorbent pad to expand to a size close to maximum, e.g. 80-95% of fully expanded, the dressing may accommodate the thereby developed stress without giving rise to folds and wrinkles of the absorbent layer. Furthermore, such dressing may be suitable for less exuding wounds or for dressing being changed more often, thus not utilizing the full absorbent capacity of the dressing.

The envelope may have a surface area of at least 85% more preferred 90% and most preferred 95% of the surface area of the absorbent pad when fully expanded.

In one embodiment of the invention the surface area of the envelope is at least 110% more preferred 120% and most preferred 130% of the surface area of the absorbent pad when fully expanded.

When reference is made to the dimensions of the absorbent pad without specifying the condition of the pad it is to be considered as the dry absorbent pad before exposure to moisture.

The surface area of the envelope may be at least 10% larger than the surface area of the absorbent pad, more preferred at least 20%, even more preferred at least 30% and most preferred at least 40% larger than the surface area of the absorbent pad.

In one embodiment of the invention the lateral dimensions of the envelope is at least 50% larger than the lateral dimensions of the absorbent pad.

Using highly expanding absorbent pad the surface area of the envelope may be more than 60%, preferably more than 70% and even more preferred more than 80% and most preferred more than 90% larger than the surface area of the absorbent pad.

In one embodiment of the invention the edges of the envelope is provided with vertical wall, thus obtaining a box-shaped envelope. The vertical walls may be folded, e.g. like an accordion providing an initially flat envelope, and when the absorbent pad inside the envelope expands the wall may rise to a vertical position. One or both of the two layers may constitute the vertical wall or it may be a third layer connecting the two layers. The embodiment with vertical walls is especially suitable for thick absorbent pads.

The backing layer may be of any suitable material known per se for use in the preparation of wound dressings e.g. a foam, a non-woven or a polyurethane, polyethylene, polyester or polyamide film or a laminate of two or more layers. The backing layer may be liquid impervious but vapor permeable or it may be of a type having a higher water permeability when in contact with liquid water than when not in contact.

In one embodiment of the invention the backing layer is permeable, such as a non-woven.

A suitable material for use as a backing layer is polyurethane. A preferred low friction film material is disclosed in U.S. Pat. No. 5,643,187.

The backing layer, which comprises the non-skin-facing sheet of the envelope, may constitute the top layer of the dressing or the dressing may be provided with one or more cover layers overlying the non-skin-facing surface of the backing layer. The cover layer may be liquid impervious but vapor permeable or impermeable thus facilitating a bacteria-proof protection layer over the dressing of the present invention.

In one embodiment of the invention the cover layer is prepared from a material providing desired surface properties such as high or low friction, thus facilitating application by a secondary dressing or reduce friction against e.g. clothes. Such material may e.g. be a non-woven, textile or a foam.

The skin-facing layer may be of any suitable material known per se for use in the preparation of wound dressings e.g. a foam, a non-woven or a polyurethane, polyethylene, polyester or polyamide film or a laminate of two or more layers.

The skin-facing layer may be permeable to liquids or it may be impermeable, e.g. water impermeable but vapor permeable. If the layer is impermeable it may be provided with one or more apertures in order to allow the wound exudates to penetrate into the absorbent pad.

A stop layer for preventing any speck or fluff from the absorbent layer to enter the wound may cover the skin-facing surface of the absorbent layer. The stop layer may be of any suitable material known in the art being capable of retaining absorbent material, such as a web or net, non-woven or a perforated polymeric film, knits, PP, PE, polyester or lycra.

At least one of the layers of the envelope may comprise one or more apertures.

The skin-facing layer may comprise an aperture in the area covering the absorbent layer. The aperture may especially be located centrally over the wound and will render it possible to have a fast absorption as the absorbent layer will be in direct contact with the exudates and the exudates will not have to pass through the skin-facing layer first.

The aperture may in one embodiment of the invention be decentrally placed. Such position of the aperture may be advantageous from dressings worn vertically, such as leg ulcers, as the gravity will lead the majority of the wound exudate to the lower portion of the dressing. Using impermeable layers for the envelope, the envelope may serve as a pocket for collecting wound exudates, and without giving rise to maceration of the skin below the wound, as the impervious skin-facing layer prevents wetting of the skin beneath the skin-facing layer. Thus, the dressing of the present invention offers additional safety in terms of low risk of maceration and/or leakage.

The apertures may be in the form of a pattern of smaller or larger apertures.

Preferably the aperture is in the form of one central aperture.

Preferably the aperture has about the same dimensions as of the wound. This will enhance the absorption rate over the wound, while the skin-facing layer will protect the fragile skin next to the wound from maceration.

The dimensions of the aperture are preferably smaller than the dimensions of the absorbent pad, in order to control the pad and facilitating the pad stays in the envelope. However, if the aperture is closed by a permeable layer such as a net, the dimensions of the aperture may if desired exceed the dimensions of the absorbent pad.

The absorbent pad may be located concentric to the aperture or it may be located excentriccally. The excentric location may be especially suitable for dressings worn vertically, such as dressings for leg ulcers, as a majority of the wound exudates, due to gravity, may enter the lower part of the absorbent pad.

The aperture in the skin-facing layer may be enlarged to adapt to the size of the wound. The enlargement may be done by scissors or by the use of pre-cuts lines in the skin-facing layer, enabling easy removal of excess skin facing layer before application. The pre-cuts lines may e.g. be in the form of concentric circles, squares, or a helix.

The skin-facing layer may be a layer of non-woven, foams, knits, Polypropylene, Polyethylene, polyester, polyurethane or Lycra. The material for the skin-facing layer may be permeable, semi-permeable or impermeable. The layer may be provided with multiple perforations in order to enhance permeability.

In one embodiment of the invention the skin-facing layer may constitute the entire skin-contacting surface.

The absorbent pad may be any suitable material for absorbing wound exudates. The absorbent pad has a surface area expansion of at least 10% more preferred at least 20%, even more preferred at least 25% and most preferred at least 30%. In one embodiment of the invention the absorbent pad has a volume expansion of at least 35%. The larger the expansion the more critical it is that the dressing is able to handle the expansion. The absorbent pad may in one embodiment have an expansion of at least 40% or even at least 50%.

The expansion is determined by measuring the surface area of an absorbent pad, immersing it into saline (0,9% NaCl) water until maximum absorption is obtained (24 hours) and then measuring the area again.

The absorbent pad is preferably in the form of a layer having a lateral extension far greater than its vertical extension, thus the expansion of volume may primary be an issue in the lateral direction and thus mostly have an impact of the surface area of the pad. However, the pad may also be quite thick, thus also expanding vertically too.

The absorbent layer may comprise any absorbent material known per se being suitable for use in wound care devices, e.g. foam, polyacrylate, CMC, cellulose or derivatives thereof, super absorbing fibers or particles, gums or alginate or mixtures thereof. The absorbent pad may be in the form of one or more layers of same or different material.

In a preferred embodiment of the invention the absorbent pad comprises foam, such as a polyurethane foam.

The absorbent pad may have an absorption capacity of at least 0,1 g/m², more preferred at least 0,2 g/m², even more preferred at least 0,3 g/m² and most preferred at least 0,4 g/m². In one embodiment of the invention the absorption capacity of the absorbent pad is at least 0,6 g/m².

The absorbent pad may be provided with incisions or slits. The incisions or slit may extend from edge portion towards the center portion or they may be homogeneously distributed over the surface of the pad. The incisions or slits may help the absorbent pad expand more freely and without bulking during the wetting of the absorbent pad.

In a preferred embodiment of the invention the absorbent pad is provided with a number of radial slits, extending from the edge portion and towards, but not through, the central portion. Thus the pad may resemble a flower with a center and petals. If the pad is wetted inhomogeneous, the “petal(s)” being affected may expand without being detained by the dry, non-expanded part of the pad. Thus internal stress in the dressing may be avoided.

The absorbent pat may comprise at least two slits; more preferred 3 and most preferred 4 slits. In one embodiment of the invention the pad comprises 5 or more slits.

In one embodiment of the invention the slits may be through-going, thus dividing the absorbent pad into two or more portions.

The surfaces of the backing layer and the skin-facing layer contacting the absorbent pad are preferably non-adhesive. This renders it possible for the absorbent pad to be free-floating in the envelope, thus facilitating free expansion of the absorbent pad when exposed to moisture.

The absorbent pad may be contained in, but not attached to, the envelope. The absorbent pad will thus be able to slide against the backing layer when expanding. If the surface area of the absorbent pad was attached to the backing layer the absorbent pad would be restricted in its expansion, leading to wrinkling and undesired pressure against the wound.

In one embodiment of the invention the absorbent layer is attached to the envelope in a restricted area. The attachment may be in the form of one or more adhesive areas or dots or in the form of weldings. The attachment being confined to a limited zone of the absorbent pad, it is still facilitated that the absorbent pad is able to expand freely in the envelope. The presence of such attachment may be advantageous for the handling of the dressing during production, as well as it secures that the absorbent pad stays in place during handling and use. Examples of such attachments may be a central or decentral adhesive dot, thus fixing the absorbent layer in the envelope during production, storage and handling of the dressing, but not limiting the ability of free expansion of the absorbent pad when wetted.

The absorbent pad may be located inside but unattached to the envelope or it may be attached to at least one of sheets of the envelope. The attachment is preferably in the form of one or more adhesive dots or point lamination. It may be advantageous to attach the absorbent pad inside the envelope in order to help it stay in place at the center of the envelope. If the absorbent pad moves towards the edge portion of the envelope, it may have difficulties to expand freely to the side being close to the edge portion. Furthermore, it may facilitate production of the dressing when the absorbent pad is immobilized.

Preferably a single point, in the center portion or excentrically, attaches the pad. This allows the pad to expand freely outwards from the attachment point. However, more than one attachment point may also be applicable, e.g. by using an adhesive or welding that detaches when wetted.

In one embodiment of the invention the backing layer is coated with a layer of adhesive, wherein the adhesive may loose it adhesive tack when wetted and thus does not inhibit the free movements of the pad in the envelope. The adhesive may be any suitable adhesive with such properties, e.g. an acrylate adhesive in a continuous or discontinuous layer.

The absorbent pad may be able to move freely in the envelope. The inner surfaces of the envelope may in one embodiment of the invention have low friction, facilitated by the choice of material or by a coating of the inner surface.

The skin-facing surface of dressing may further comprise an adhesive layer for attaching the dressing to the skin. The adhesive layer may be a continuous or discontinuous layer or the adhesive layer may be coated in a pattern. In a preferred embodiment of the invention the adhesive layer comprises one or more central apertures, e.g. where the adhesive layer is located at the border portion of the dressing, thus providing an adhesive flange surrounding a non-adhesive central part.

In one embodiment of the invention the dressing comprises a layer of a low-tack adhesive, such as a silicone adhesive. The layer may be continuous or discontinuous in the form of a pattern.

The adhesive may be any skin-friendly adhesive known per se, e.g. an adhesive comprising hydrocolloids or other moisture absorbing constituents for prolonging the time of use. The use of a hydrocolloid adhesive may provide an excellent protection of the surrounding skin of the wound by inducing the moist wound healing environment, and yet avoiding maceration.

The skin facing layer may also comprise any other adhesives, preferably pressure sensitive adhesives and/or hot-melts, chosen from a wide range of different types of adhesives for instance the acrylic types, and types derived from PIB, polyurethanes, EVA-compounds, APAO's, silicones, polyvinyl ether.

The adhesive surface of the dressing may be protected by a protective cover or a release-liner before application. The protective cover or release liner will typically be siliconised thermoplastic films based on for example polyolefins such as polyethylene, polypropylene, PET or the like or it may be siliconised paper.

The dressing according to the invention may comprise an active ingredient.

The wound dressing according to the invention may comprise one or more active ingredients, e.g. a pharmaceutical medicament. This opens for a combined medical treatment of a wound, where the dressing absorbs wound exudate and the pharmaceutical medicaments will be applied to the wound. The pharmaceutical medicaments will either be incorporated in the wound dressing or migrate to the wound surface and promote its function.

Examples of such pharmaceutical medicaments includes a cytochine such as a growth hormone or a polypeptide growth factor, bacteriostatic or bactericidal compounds, e.g. silver salts such as sulphadiazine, silver nitrate, silver acetate, silver lactate, silver sulphate, silver sodium thiosulphate, silver-zirconium complexes or silver chloride, zinc or salts thereof, metronidazol, sulpha drugs, and penicillin's, tissue-healing enhancing agents, vitamins such ascorbic acid, enzymes for cleansing of wounds, e.g. pepsin, papain, trypsin and the like, proteinase inhibitors or metalloproteinase inhibitors, elastase inhibitors and/or other therapeutic agents which optionally may be used for topical application, pain relieving agents such as ibuprofen, lidocaine or chinchocaine, emollients, retinoids or agents having a cooling effect which is also considered an aspect of the invention.

The active ingredient may also comprise odor controlling or odor reducing material.

The skin-facing layer may be impregnated with zinc-paste or other skin conditioning or healing enhancing materials.

A dressing according to the invention is, after application, in the form of a single unit, not an assembly. The two layers of the envelope are unreleasably sealed to each other and the dressing may not be opened from the top as the reopenable dressings known in the art may do. A dressing in a single unit decrease the risk of leakage compared to an assembled or reopenable dressing.

The dressing may be of any suitable shape, e.g. rounded, rectangular or other geometric shape, or it may have a shape adapted for the body part to which it may be applied, e.g. the sacrum.

### DESCRIPTION OF THE PREFERRED EMBODIMENTS

The invention is now explained more in detail with reference to the drawings showing preferred embodiments of the invention.

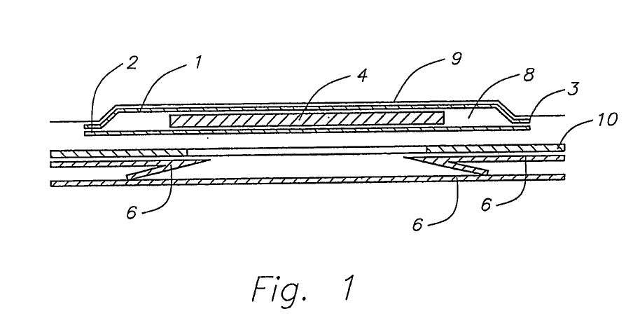

FIG. 1 discloses an exploded view of an embodiment of the invention comprising a backing layer (1) and a skin-facing layer (2); the two layers being sealed along their periphery (3) in order to produce an envelope. In the envelope, between the two layers (1, 2) is placed an absorbent pad (4). The backing layer (1) and the skin-facing layer (2) may be prepared from water impervious but vapor permeable film such as a polyurethane film and welded together along the edge portion (3). The absorbent pad (4) is preferably a foam, such as a polyurethane foam. The skin-facing layer may be provided with an aperture (not shown) for enabling a close contact between the wound and the absorbent pad (4). The size of the aperture may be adapted to the size of the wound, e.g. by cutting with a pair of scissors. In one embodiment of the invention the aperture may comprise a permeable layer such as a net for controlling the absorbent layer (4) and avoiding the dressing from sticking to the wound. The dressing of the invention may further be provided with an adhesive flange (10) for attaching the dressing to the skin. The skin-facing adhesive surface is protected by one or more release liners (6) before application. The size of the envelope exceeds the size of the absorbent pad (4) leaving an amount of spare room (8). When the absorbent pad (4) absorbs wound exudates it will expand into the spare room (8) instead of expanding vertically into buckles as it would if the lateral expansion possibilities were limited. The dressing may further be provided with a cover layer (9), such as a non-woven in order to obtain a smooth and comfortable surface.

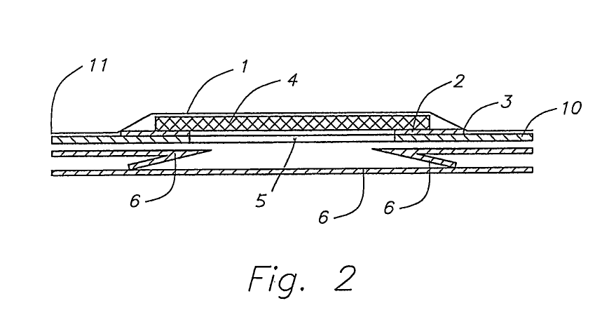

FIG. 2 shows another embodiment of the present invention wherein the backing layer (1) extends further than the skin-facing layer (2). The two layers are sealed (3) along the periphery of the skin-facing layer (2). The absorbent pad (4) is located in the envelope. The skin-facing layer (2) comprises an aperture (5) enabling direct contact to the absorbent layer (4). The skin-facing layer (2) and the skin-facing portion of the backing layer (11) not being covered by the skin-facing layer (2) are coated with an adhesive layer (10). The adhesive surface is optionally protected by one ore more release liners (6) to be removed before application.

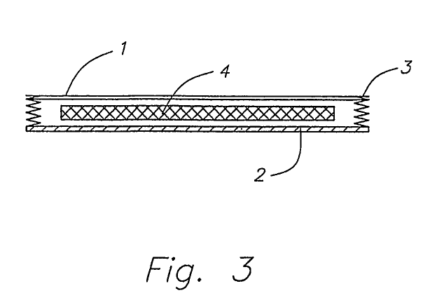

FIG. 3 shows a non-adhesive embodiment of the dressing of the invention. The dressing comprises a backing layer (1) and a skin-facing layer (2) sealed along the periphery of the layers (3). The backing layer (1) may be an impermeable film and the skin-contacting layer (2) may be a non-woven or a net being permeable to wound exudates.

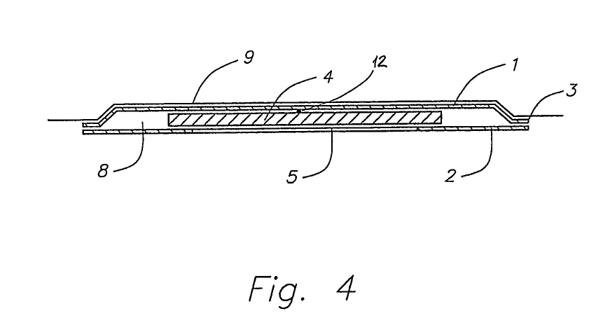

FIG. 4 shows a preferred embodiment of the present invention comprising a backing layer (1) and a skin-facing layer (2) welded together along the edge portion (3) to form an envelope and an absorbent pad (4) contained in the envelope. The skin-facing layer (2) comprises a central (5) aperture enabling direct contact between the absorbent pad (4) and the wound. The skin-facing layer (2) of the envelope may be prepared from a laminate, comprising a polyester non-woven closest to the skin for pleasant skin feeling, then a water impervious but vapor permeable film, and then another polyester non-woven layer. The second non-woven layer provides a smooth surface against the absorbent pad (4) facilitating the sliding/floating of the expanding absorbent pad when wetted. The backing layer (1) may be prepared from the same laminate. A laminate may provide a certain rigidity, which may be advantageous as it may smoothen out the edge portions of the absorbent pad, and thus reduce the risk of pressure marks from this. An example of a laminate suitable for the layer is Saranex 630 film, which is a multilayer structure that contains the following polymers: Chlorinated polyethylene (CPE), polyethylene/vinyl acetate (EVA) and polyvinylidene chloride/vinyl chloride (PVdC/PVC). The film is plasticized with epoxidized soybean oil (<1%). Preferably the absorbent pad (4) is attached in one or more points (12) to the backing layer of the envelope.

1. A wound dressing comprising a backing layer and a skin facing layer and an absorbent pad, wherein the absorbent pad is sandwiched between the backing layer and the skin facing layer, and the two layers constitutes an envelope, the absorbent pad has an expansion of surface area, when fully expanded, of at least 10%, and wherein the surface area of said envelope is at least 10% larger than the surface area of the non-expanded absorbent pad.

2. A wound dressing according to claim 1 wherein the surface area of the envelope is at least 20% larger than the surface area of the absorbent.

3. A wound dressing according to claim 1 wherein the surface area of the envelope is at least 30% larger than the surface area of the absorbent.

4. A wound dressing according to claim 1 wherein the surface area of the envelope is at least 40% larger than the surface area of the absorbent.

5. A wound dressing according to claim 1 wherein the surface area of the envelope is at least 50% larger than the surface area of the absorbent.

6. A wound dressing according to claim 1 wherein the dressing comprises an adhesive layer.

7. A wound dressing according to claim 1 wherein the adhesive layer is located at the border portion of the dressing.

8. A wound dressing according to claim 1 wherein the skin-facing layer comprises non-woven, foams, knits, films, polypropylene, polyethylene, polyester, polyurethane or Lycra or mixtures or laminates thereof.

9. A wound dressing according to claim 1 wherein the skin facing layer comprises an aperture.

10. A wound dressing according to claim 1 wherein the skin facing layer is impermeable to water.

11. A wound dressing according to claim 1 wherein the absorbent pad comprises foam.

12. A wound dressing according to claim 1 wherein the absorbent pad comprises incisions or slits.

13. A wound dressing according to claim 1 wherein the absorbent pad is attached to at least one of layers of the envelope.

14. A wound dressing according to claim 1 wherein the dressing comprises a cover layer overlying the non-skin-facing surface of the backing layer.
